(19) United States
(12) Patent Application Publication
(10) Pub. No.: US 2018/0282687 A1
(43) Pub. Date: Oct. 4, 2018

(54) Propagation of Yeast for Removal of Sugars From Spent Sulfite Liquor

(71) Applicant: Domtar Paper Company, LLC, Fort Mill, SC (US)

(72) Inventor: Neelakantam NARENDRANATH, Fort Mill, SC (US)

(21) Appl. No.: 15/938,881

(22) Filed: Mar. 28, 2018

Related U.S. Application Data
(60) Provisional application No. 62/477,559, filed on Mar. 28, 2017.

(51) Int. Cl. C12N 1/24; C12N 1/18; C12N 1/16; C08H 7/00

(52) U.S. Cl. CPC C12N 1/24; C08H 6/00; C12N 1/16; C12N 1/18

(57) ABSTRACT

Methods for production of yeast biomass and removal of sugar from red liquor by propagating yeast on sugars in red liquor byproduct of the sulfite pulping process are disclosed. Yeast are propagated using the sugars in red liquor as a carbon source, thereby consuming the sugars and producing a composition of lignosulfonates that has a reduced sugar content. Disclosed methods allow for separation of sugars from lignosulfonates in red liquor without costly ultrafiltration or nanofiltration processes and also produce valuable yeast biomass.

### CROSS REFERENCE TO RELATED APPLICATIONS

This application claims benefit of priority to U.S. Provisional Patent Application No. 62/477,559 filed Mar. 28, 2017, which is incorporated by reference in its entirety.

### FIELD OF INVENTION

The present invention relates to systems and methods for separating sugars from lignosulfonates in spent sulfite liquor (also known as red liquor) by using the sugars as a carbon source for yeast propagation.

### BACKGROUND

Red liquor, or spent sulfite liquor, is a byproduct from the sulfite pulping process. While sulfate pulping, or kraft pulping, is the predominant pulping process today, sulfite pulp remains an important commodity for specialty papers. During the sulfite pulping process, a large portion of the lignin and the majority of the hemicellulose in wood get dissolved into the cooking liquor (red liquor). Unlike in the black liquor from the kraft process, the hemicelluloses in the red liquor from the sulfite process are hydrolyzed into monomers. This makes red liquor a potentially good feedstock for ethanol production since the sugars are already in a fermentable monomeric form. Organisms that are able to metabolize C5 sugars such as xylose and/or arabinose have been used for ethanol production from red liquor.

The calcium lignosulfonates in red liquor have a variety of uses, including as a carrier for fat-soluble vitamins, carotenoids and other functional ingredients. Lignosulfonates are also used for vanillin production, which can be used both as a flavoring agent and as a chemical building block for the synthesis of bio-based polymers. In order to be used in these ways, the lignosulfonates must be separated from the sugars in red liquor.

As the sulfite pulping process is currently configured, removal of sugars from the red liquor is not possible during the pulping process itself. It is possible to remove sugars from the red liquor left after the pulping process is complete, but doing so requires additional processes beyond the pulping process, such as ultrafiltration and/or nanofiltration of the red liquor, which require additional equipment and expense. Normally, the removed sugars go to waste, although use of the sugars for ethanol production has been reported. However, using the separated sugars for ethanol production would require even more expense, including for installation of fermentation and distillation equipment. The ability to produce relatively low-value biofuels from the separated sugars does not economically justify the expense of separating the sugars from the red liquor.

A more economically valuable product that could be made using the sugars in red liquor would be food-grade yeasts. Yeasts have been propagated using sugars in black liquor from soda pulping as a carbon source (Hu et al., BioResources 10:3908-21 (2010)). However, the prior art methods involving propagation of yeast in black liquor do not produce a sugar-free or reduced sugar lignosulfonate composition suitable for beneficial uses like production of vanillin, but instead merely prepare the black liquor for waste disposal by reducing its chemical oxygen demand. There exists a need for an economically efficient process of producing sugar-free lignosulfonate compositions from red liquor.

### SUMMARY

This disclosure includes a solution to the economically inefficient handling of the red liquor byproduct of the sulfite pulping process. The solution involves using yeast propagation to consume the sugars in red liquor. This can result in the production of economically valuable food grade yeasts while at the same time separating the sugars away from lignosulfonates in the red liquor. The lignosulfonates can then be used for various purposes, including production of vanillin. The inventors have discovered the appropriate culture conditions for making use of the sugars in the red liquor with minimal to no production of ethanol and for removing sugars from lignosulfonates without expensive filtration processes. The methods disclosed herein thus improve the economic efficiency of the handling of red liquor and of the sulfite pulping process as a whole.

In some embodiments of the present methods of propagating yeast, the method comprises: (a) combining an aqueous red liquor byproduct from a sulfite pulping process and a first cell mass of yeast to form a liquid culture, wherein the red liquor comprises monosaccharides and lignosulfonates; and (b) incubating the liquid culture for sufficient time period to form a second cell mass of yeast that has a higher dry cell weight than the first cell mass of yeast; wherein no more than 0.1 g/L of ethanol is produced in the liquid culture during step (b). The minimal production of ethanol allows for more efficient production of yeast biomass during the incubation step, which provides for a more economically valuable product from yeast propagation compared to methods that produce substantial amounts of ethanol. In some embodiments, substantially no ethanol is produced by the yeast during step (b). Substantially no ethanol is produced if no more than trace amounts of ethanol can be detected in the liquid culture after the incubation is complete. In some embodiments, the pH of the red liquor is adjusted to about 5.5, or the pH of the red liquor is confirmed to be about 5.5, before or after being combined with the first cell mass of yeast. In some embodiments, the pH of the liquid culture is maintained between about 4 and 6 or between about 5.3 and 5.7.

In some embodiments of the present methods of propagating yeast, the first cell mass of yeast has a dry cell weight of about 0.05 to 0.15 g per liter of liquid culture. Incubation during step (b) can cause the first cell mass of yeast to propagate and produce a second cell mass of yeast. The second cell mass of yeast may be several fold larger, as measured by either dry cell weight of yeast or yeast cell number. In some embodiments, the second cell mass of yeast has a dry cell weight of approximately 5 to 15 g per liter of liquid culture. In some embodiments, the second cell mass has a dry cell weight approximately 50 to 100 times that of the first cell mass of yeast. In some embodiments, the yeast consume at least 90, 95, or 99% of the monosaccharides in the liquid culture. In some embodiments, the chemical oxygen demand of the red liquor and/or liquid culture is reduced by at least 80, 90, 95, or 99% during step (b). In some embodiments, the concentration of monosaccharides in the red liquor before incubation is in the range of 24 to 35 g/L. In some embodiments, the concentration of monosaccharides in the liquid culture after incubation is less than 2, 1, 0.5, or 0.1 g/L. In some embodiments, the yield of yeast resulting from the incubation step is 0.45 to 0.55 grams of dry cell weight per gram of monosaccharides consumed by the yeast.

In some embodiments of the present methods of propagating yeast, the monosaccharides in the red liquor are primarily pentose sugars. In some embodiments, at least 70, 80, 90, or 95% of the monosaccharides in the red liquor are pentose sugars. In some embodiments, the pentose sugars comprise xylose. In some embodiments, the red liquor is a byproduct of sulfite pulping of hardwoods. In some embodiments, the red liquor is a byproduct of sulfite pulping of softwoods. In some embodiments, the monosaccharides in the red liquor are the only carbon source for yeast propagation present in the liquid culture.

In some embodiments of the present methods of propagating yeast, the yeast are Saccharomyces cerevisiae, which may be wild-type or may be engineered to have certain genetic modifications. The genetic modifications may, for example, expand the range of monosaccharides that the yeast can metabolize and use as a carbon source to support propagation and biomass growth. In some embodiments, the Saccharomyces cerevisiae have been modified to be able to metabolize and grow on xylose. In some embodiments the yeast are a type of yeast that can naturally metabolize and grow on pentose sugars, including xylose. In some embodiments, the yeast are Candida utilis.

In some embodiments of the present methods of propagating yeast, the liquid culture is incubated for 12 to 36 hours. The culture may be maintained at a temperature in the range from about 25 to 37° C. or in the range from about 30 to 32° C. In some embodiments, step (b) comprises agitating the liquid culture at 300 to 750 rpm. In some embodiments, step (b) comprises aerating the liquid culture at a rate of between 0.5 and 2 volumes of air per volume of medium per minute (vvm). The liquid culture may further comprise a nutrient source.

In some embodiments of the present methods of propagating yeast, the method further comprises separating the lignosulfonates in the liquid culture from yeast in the liquid culture. The yeast that are separated can include the first cell mass of yeast and the second cell mass of yeast. The separation can be accomplished by, for example, filtration or centrifugation. In some embodiments, the filtration employs a membrane that allows the lignosulfonates to pass through but does not allow the yeast to pass through. In some embodiments, the method excludes any filtration step that would allow monosaccharides to pass through while not allowing lignosulfonates in the red liquor and/or liquid culture to pass through. In some embodiments, the method excludes any ultrafiltration or nanofiltration.

In some embodiments of the present methods of removing monosaccharides from a lignosulfonate composition, the method comprises: (a) obtaining an aqueous red liquor byproduct of a sulfite pulping process, wherein the red liquor comprises monosaccharides and lignosulfonates; (b) propagating yeast in the red liquor, wherein the yeast metabolize the monosaccharides; and (c) removing the yeast from the red liquor, thereby producing a lignosulfonate composition comprising lignosulfonates, wherein the concentration of monosaccharides in the lignosulfonate composition is no more than 0.1 g/L. In some embodiments, the removal of monosaccharides from the lignosulfonates in the red liquor is accomplished without the use of a filtration step that employs a membrane that allows monosaccharides to pass through but does not allow the lignosulfonates to pass through. In some embodiments, the removal of monosaccharides from the lignosulfonates in the red liquor is accomplished without the use of ultrafiltration or nanofiltration. In some embodiments, the dry cell weight of the yeast increases by at least 50-fold during step (b). In some embodiments, the other features of the method of propagating yeast that are described above can also be incorporated into this method of removing monosaccharides from a lignosulfonate composition. A person skilled in the art will recognize that the features described in one method can readily be incorporated into other methods disclosed herein.

In some embodiments of the present methods of removing monosaccharides from a lignosulfonate composition, the lignosulfonate composition that results from the methods described herein in which sugars and yeast have been removed from the lignosulfonates in red liquor is suitable for use in production of vanillin or for use in various other economically beneficial downstream applications.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Effective” or any variation of this term, when used in the claims or specification, means adequate to accomplish a desired, expected, or intended result.

The terms “about” or “approximately” are defined as being close to as understood by one of ordinary skill in the art.

The methods disclosed herein can “comprise,” “consist essentially of,” or “consist of particular components, compositions, ingredients, etc. disclosed throughout the specification.

Other objects, features and advantages of the present invention will become apparent from the following figures and detailed description. It should be understood, however, that the figures and detailed description, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 shows an illustration of an embodiment of a method of propagating yeast in red liquor to consume sugars and produce yeast biomass.

### DETAILED DESCRIPTION OF ILLUSTRATIVE EMBODIMENTS

The methods disclosed herein efficiently remove sugars from lignosulfonates in red liquor and produce valuable yeast biomass using the sugars from red liquor as a carbon source. These and other aspects of the disclosed method will be described in greater detail below.

### A. Sulfite Pulping and Red Liquor

The sulfite pulping process produces wood pulp, which is almost pure cellulose fibers, by extracting the lignin from wood chips. The process involves cooking, or digesting, wood chips at high temperature and pressure in an acidic solution of various salts of sulfurous acid. The salts used in the pulping process are either sulfites (SO₃²⁻) or bisulfites (HSO₃⁻), depending on the pH. After cooking is complete, the spent cooking liquor, referred to as red liquor, is separated from the pulp.

In the cooking process, the hemicellulose in the wood is hydrolyzed into monomer sugars (i.e., monosaccharides). The composition of the monosaccharides in the red liquor depends on the type of wood that is used. Red liquor from pulping coniferous softwoods contain a relatively high proportion of hexoses, while red liquor from pulping deciduous hardwoods contain a relatively high proportion of pentoses (mainly xylose). As an example, sugar analysis of red liquor from a sulfite mill has showed that xylose was the predominant sugar and was present at a concentration of about 23.5 g/L. Minor quantities of glucose, mannose, and arabinose were also present. The total monosaccharide content of red liquor is typically in the range of 24 to 27 g/L and can also be as high as 35 g/L or higher. In some embodiments, the total monosaccharide content of the red liquor is about 10, 15, 20, 25, 30, 35, or 40 g/L or is between any two of those values. In some embodiments, the proportion of monosaccharides that are pentoses is at least about or is about 75, 80, 85, 90, 95, or 99% or is between any two of these values.

In addition to sugars, red liquor includes lignosulfonates. The lignosulfonates are produced during cooking by the addition of sulfonate groups onto lignin, which makes the lignin soluble in water, allowing it to be separated from the cellulose in the wood. The lignosulfonates can be present in red liquor at a concentration in the range of about 40 to 45% by weight on a dry solids basis, meaning that the dry weight of the lignosulfonates in the red liquor is about 40 to 45% of the total dry solids content of the red liquor. In some embodiments, the concentration of lignosulfonates in the red liquor is about 20, 25, 30, 35, 40, 45, 50, 55, or 60% by weight on a dry solids basis or is between any two of these values.

Lignosulfonates in red liquor have many potential uses, including but not limited to chemicals, battery expanders, bypass protein, carbon black dispersions, cement, ceramics, concrete admixtures, emulsions, fertilizers, gypsum board, humic acid, industrial binders, industrial cleaners & water treatment additives, soil conditioners, micronutrients, mining and mineral processing, oil field chemicals, pelleting performance enhancers, and road & soil dust control, among others. Methods disclosed herein can be used as part of the process of preparing lignosulfonates to be used in these ways.

Methods disclosed herein can be used to remove monosaccharides from lignosulfonates in red liquor. The monosaccharides can be consumed by yeast, and the yeast can be harvested, leaving a lignosulfonate composition that has a reduced content of monosaccharides or that has no appreciable amounts of monosaccharides. In some embodiments, propagating yeast using the monosaccharides in red liquor results in a reduction of the monosaccharide content in the red liquor by at least about or about 50, 55, 60, 65, 70, 75, 80, 85, 90, 95, 99, or 99.9%, or between any two of these values. In some embodiments, the concentration of monosaccharides in the composition after propagating yeast and removing the yeast is at most about or is about 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 g/L or is between any two of those values.

In some embodiments of the methods disclosed herein, the yeast may be added directly to an undiluted and untreated red liquor solution that is a raw byproduct of the sulfite pulping process. In some embodiments, the red liquor solution is diluted in the yeast culture with water, growth medium, or some other liquid. In some embodiments, the red liquor is pretreated to make it more conducive for supporting growth of yeast. For example, the red liquor may be treated to remove components that may inhibit yeast growth. Pretreatment steps may be tailored to the unique properties of the particular red liquor being used. In some embodiments, the monosaccharides from the red liquor are the only source of carbon to support yeast growth. In some embodiments, a culture may be supplemented with other sugars and nutrients that can support yeast growth.

### B. Yeast

Methods disclosed herein employ yeast to consume sugars in red liquor. Propagation of yeast using the red liquor sugars as a carbon source has multiple benefits, including the production of yeast biomass and the production of a lignosulfonate composition that has reduced monosaccharide content or is free of monosaccharides.

Some yeast species, such as Candida and Saccharomyces species, have been employed as producers of microbial protein, referred to as single-cell protein (SCP), to convert agro-industrial wastes, such as effluents from paper and olive mills into a valuable amino acid supplement for animal feeds and plant nutrients. Candida utilis (also known as torula yeast) is classified as one of the most promising microorganisms for its protein content, which can account for up to 50% of the yeast's dry weight, and for the vitamins, minerals, and other nutrients present in the yeast. Candida utilis has a relatively high concentration of essential amino acids in its proteins and possess the ability to metabolize a wide range of saccharides.

Many different yeasts can be propagated using the sugars in red liquor as a carbon source according to the methods disclosed herein. Any yeast that is able to use the sugars present in the red liquor as a carbon source can be used. Red liquor may contain varying amounts of hexose and pentose monosaccharides. Some yeast have a narrow range of monosaccharides that they can use for growth. For example, some yeast are unable to use pentose sugars for growth but are able to use hexose sugars. A combination of different yeast species may be used to broaden the range of monosaccharides that can be consumed during incubation of the yeast in a culture fed with the red liquor.

Some embodiments of the methods disclosed herein employ yeasts that are naturally able to use xylose as a carbon source for growth. Some of these yeasts include but are not limited to Candida utilis, Candida sonorensis, Candida chilensis, Candida entomophila, Candida insectamans, Candida intermedia, Candida lodderae, Candida maltose, Candida steatolytica, Candida succiphila, Candida torresii, Candida tropicalis, Candida viswanathii, Pichia guilliermondii, Pichia naganishii, Pichia sargentensis, Pichia pastoris, Kluyveromyces marxianus and other Candida, Pichia, and Kluyveromyces species.

Some yeast species that cannot naturally use a given sugar as a carbon source for growth can be engineered to expand the range of sugars that they can use. For example, wild-type strains of Saccharomyces cerevisiae are unable to use pentose sugars such as xylose as a carbon source for growth. However, they may be genetically modified by well-known processes to use xylose. Many other yeast with genetic modifications expanding the range of sugars that can be metabolized are known.

In some embodiments, the yeast are inoculated in a liquid culture at a concentration of 0.01 to 10 g of dry cell weight per liter of the culture. In some embodiments, the concentration is between about 0.02 and 5 g of dry cell weight per liter of the culture. The yeast inoculated into the culture is referred to herein in some instances as a first cell mass of yeast. In some embodiments, the first cell mass of yeast has a concentration of at least about, at most about, or about 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 g dry cell weight per liter of culture, or between any two of these values.

Propagating the yeast according to the methods described herein causes the yeast biomass in the culture to increase efficiently. In some embodiments, incubating the culture for a time between 12 and 36 hours causes the yeast biomass to increase by at least 50 or 100-fold with no or minimal production of ethanol (e.g., less than 0.1 g/L of ethanol). In some embodiments, some amount of ethanol is produced as the yeast propagates. In some embodiments, the increase in yeast biomass is at least about 10, 15, 20, 25, 30, 35, 40, 45, 50, 55, 60, 65, 70, 75, 80, 85, 90, 95, 100, 105, 110, 115, 120, 125, 130, 135, 140, 145, or 150-fold or is between any two of these values. In some embodiments, these increases in biomass may be accomplished with incubation times of about 12, 24, 36, or 48 hours, or between any two of these values. The yeast biomass produced during the incubation of the culture is referred to herein in some instances as the second cell mass of yeast. In some embodiments, after incubating the culture, the second cell mass of yeast has a concentration of at least about, at most about, or about 5, 10, 15, 20, 25, or 30 g dry cell weight per liter of culture, or between any two of these values. In some embodiments, these increases in yeast biomass are accomplished without the production of detectable amounts of ethanol. In some embodiments, these increases in yeast biomass are accomplished with production of ethanol that results in a concentration of no more than 0.01, 0.05, 0.1, 0.15, 0.2, 0.25, 0.3, 0.35, 0.4, 0.45, or 0.5 g/L in the culture.

### C. Propagation Conditions

The conditions at which the yeast are propagated are chosen so as to ensure efficient growth of yeast biomass and consumption of the monosaccharides in red liquor. Conditions may also be tailored to be conducive to efficient yeast biomass growth without inducing production of substantial amounts of ethanol. Important parameters include the temperature, pH, and aeration rate.

In some embodiments, the temperatures is in a range from 20 to 40° C., from 25 to 37° C., or from 30 to 32° C. In some embodiments, the temperature is at least about, at most about, or is about 20, 21, 22, 23, 24, 25, 26, 27, 28, 29, 30, 31, 32, 33, 34, 35, 36, 37, 38, 39, or 40° C. or is between any two of these values.

The culture can be incubated at the chosen temperature for sufficient time to allow for a predetermined amount of yeast biomass production. In some embodiments, the culture is incubated for 12 to 24 hours. In some embodiments, the culture is incubated for about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, or 72 hours or for an amount of time in a range between any two of these values.

In some embodiments, the pH of the culture is in a range from about 4.0 to 6.0, from about 4.5 to 5.5, or from about 5.0 to 5.2. In some embodiments, the pH is at least about, at most about, or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6, or is between any two of these values.

Aeration of the culture ensures that sufficient oxygen is available to the growing yeast to support respiratory growth and avoid anaerobic fermentation. Aeration can be provided by any well-known apparatus such as an air sparging system. In some embodiments, the culture is aerated at a rate of between 0.5 and 2 volumes of air per volume of medium per minute (vvm). In some embodiments, the aeration rate is about 0.5, 0.6, 0.7, 0.8, 0.9, 1.0, 1.1, 1.2, 1.3, 1.4, 1.5, 1.6, 1.7, 1.8, 1.9, or 2.0, or between any two of these values. Aeration (oxygen transfer) can also be enhanced by agitation of a growing culture. In some embodiments, the culture is grown in a flask or other container that is agitated at a speed between about 300 and 750 rpm.

Yeast cultures employed in the methods disclosed herein may additionally include nutrient sources to support growth of yeast biomass. The nutrient source can include, for example, yeast extract, urea, diammonium phosphate, magnesium sulfate, zinc sulfate, and the like.

### D. Propagation System

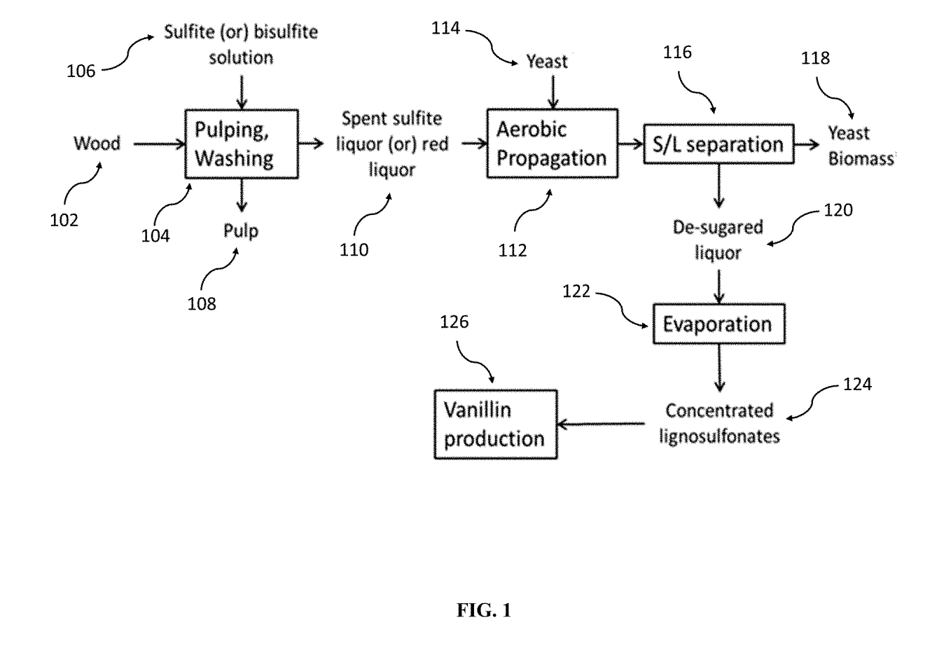

An embodiment of a method of making yeast biomass and vanillin from wood is illustrated in FIG. 1. The methods described herein can be employed as part of a method that starts with wood 102, which may be any type of hardwood or softwood. In a sulfite pulping process 104, wood 102 is mixed with a sulfite or bisulfite solution 106. During the sulfite pulping process 104, the wood 102 and sulfite or bisulfite solution 106 are heated under pressure, thereby removing lignin from the wood 102. The resulting cellulosic pulp 108 is then separated from the spent sulfite or bisulfite solution 106, which is now red liquor 110. The red liquor 110 is then used in a process of aerobic propagation 112 of yeast 114. The yeast 114 may be any yeast or combination of yeast capable of consuming the monosaccharides present in the red liquor 110. During aerobic propagation 112, the yeast 114 multiply by consuming sugars in the red liquor 110. Additional nutrients and other substances may be added to the red liquor 110 to aid in the aerobic propagation process 112, including additional nutrients to support yeast growth. The combination of red liquor 110 and yeast 114 is referred to herein in some instances as a liquid culture, which may also include additional nutrients.

Aerobic propagation 112 may continue for several hours or days, typically between 12 and 36 hours. Growth of the yeast 114 may be monitored by, for example, taking optical density readings of the culture, and sugar content of the culture may be monitored, for example, by HPLC. After aerobic propagation 112 has continued for a sufficient time to consume a desired amount of sugar and/or produce a desired amount of yeast 114, the solids (consisting primarily of yeast 114) and liquids are separated in a process of S/L separation 116 (i.e., solid/liquid separation). S/L separation 116 may be accomplished, for example, by centrifugation or filtering the culture through a filter that allows liquids and dissolved molecules to pass through, but does not allow yeast 114 to pass through. The harvested yeast biomass 118 can then be used in a variety of ways, as discussed herein. The liquid that remains after S/L separation 116 is a de-sugared liquor 120, which contains less sugar than the original red liquor, and may contain no appreciable amounts of monosaccharides. The de-sugared liquor 120 is then subjected to evaporation 122, resulting in concentrated lignosulfonates 124, which can be used in a variety of ways, including in vanillin production 126.

### E. Exemplary Yeast Propagation Method

The following procedure may be followed to propagate yeast and reduce the sugar content of red liquor. An inoculum of Saccharomyces cerevisiae that has been engineered to be capable of metabolizing xylose is grown in yeast extract, peptone, dextrose medium (YEPD) supplemented with xylose at 10 g/L for approximately 19 hours with agitation to produce a late stationary phase culture to use for inoculating the red liquor culture solution at a concentration of 0.1 grams of yeast per liter of red liquor culture solution. The red liquor culture solution is made by obtaining red liquor from a sulfite pulping process and adjusting the pH to 5.5 using potassium hydroxide and adding yeast extract (5 g/L), ammonium phosphate (4.3 g/L), magnesium sulfate (0.5 g/L), zinc sulfate (0.03 g/L), penicillin G (2 ppm), and gentamycin (2 ppm) (in some embodiments involving larger-scale production methods, antibiotics will not be used). The inoculated red liquor culture is incubated in a 2 liter bioreactor for approximately 24 hours at a temperature of 30° C., an aeration rate of 1 vvm, and an agitation rate of 450 rpm. Samples are withdrawn periodically throughout the incubation time and analyzed for dry cell weight by an oven drying method and for sugar content using HPLC equipped with the Bio-Rad HPX-87H column to measure xylose, glucose, acetic acid, and ethanol. The lignosulfonates may also be analyzed by UV spectrophotometry. It is expected that the yeast dry cell weight will be 11 to 16 g/L after 24 hours. It is expected that after 24 hours, the total sugar content of the red liquor culture will be less than 0.1 or 0.01 g/L, the xylose content will be less than 0.1 or 0.01 g/L, the glucose content will be less than 0.02 g/L, the acetic acid content will be less than 2 g/L, and the ethanol content will be less than 0.1 g/L. It is expected that the lignosulfonate analysis will reveal no change in concentration during the incubation (i.e., the lignosulfonates in the red liquor will not degrade).

1. A method of propagating yeast comprising:
(a) combining an aqueous red liquor byproduct from a sulfite pulping process and a first cell mass of yeast to form a liquid culture, wherein the red liquor comprises monosaccharides and lignosulfonates; and
(b) incubating the liquid culture for sufficient time period to form a second cell mass of yeast that has a higher dry cell weight than the first cell mass of yeast;
wherein no more than 0.1 g/L of ethanol is produced in the liquid culture during step (b).

2. The method of claim 1, wherein the concentration of monosaccharides in the red liquor is in the range of 24 to 35 g/L.

3. The method of claim 1, wherein after step (b) the dry cell weight of yeast in the liquid culture is in the range of 5 to 15 g/L.

4. The method of claim 1, wherein after step (b) the concentration of monosaccharides in the liquid culture is no greater than 0.1 g/L.

5. The method of claim 1, wherein the yeast are capable of metabolizing at least one pentose sugar.

6. The method of claim 5, wherein the yeast are Saccharomyces or Candida yeast.

7. The method of claim 1, wherein during step (b), the concentration of monosaccharides in the liquid culture is reduced by at least 95%.

8. The method of claim 1, further comprising separating the lignosulfonates from yeast in the liquid culture.

9. The method of claim 1, wherein substantially no ethanol is produced in step (b).

10. A method of removing monosaccharides from a lignosulfonate composition, the method comprising:
(a) obtaining an aqueous red liquor byproduct of a sulfite pulping process, wherein the red liquor comprises monosaccharides and lignosulfonates;
(b) propagating yeast in the red liquor, wherein the yeast metabolize the monosaccharides; and
(c) removing the yeast from the red liquor, thereby producing a lignosulfonate composition comprising lignosulfonates, wherein the concentration of monosaccharides in the lignosulfonate composition is no more than 0.1 g/L.

11. The method of claim 10, wherein monosaccharides are not removed from the red liquor by ultrafiltration or nanofiltration.

12. The method of claim 10, wherein the dry cell weight of the yeast increases by at least 50-fold during step (b).

13. The method of claim 10, wherein the concentration of the monosaccharides in the red liquor is from 24 to 35 g/L.

14. The method of claim 10, wherein the dry cell weight of the yeast in the red liquor is in the range of 5 to 15 g/L after step (b).

15. The method of claim 10, wherein the concentration of ethanol in the lignosulfonate composition is less than 0.1 g/L.
